(19) United States
(12) Patent Application Publication
(10) Pub. No.: US 2026/0040754 A1
(43) Pub. Date: Feb. 5, 2026

(54) DETECTION DEVICE

(71) Applicant: Japan Display Inc., Tokyo (JP)

(72) Inventors: Mamoru DOUYOU, Tokyo (JP); Atsunori OYAMA, Tokyo (JP)

(21) Appl. No.: 19/355,176

(22) Filed: Oct. 10, 2025

Related U.S. Application Data
(63) Continuation of application No. PCT/JP2024/013415, filed on Apr. 1, 2024.

(30) Foreign Application Priority Data
Apr. 14, 2023 (JP) 2023-066374

(51) Int. Cl. H10K 39/32; A61B 5/1172; G06V 40/13; G06V 40/145; G06V 40/40

(52) U.S. Cl. CPC H10K 39/32; A61B 5/1172; G06V 40/1318; G06V 40/45; G06V 40/145

(57) ABSTRACT

A detection device includes: an active area including an active layer of a photodiode; a coupling area provided with a coupling part at an end of a first substrate; a peripheral area between the active area and the coupling area; a sealing film sealing the active area and the peripheral area; and a first wiring line that couples a lower electrode to the coupling part. The peripheral area includes: a first portion that includes a first insulating layer, the sealing film, a second insulating layer, and a second substrate on the first substrate; and a second portion in which at least one of the sealing film, the second insulating layer, and the second substrate is removed compared with the first portion. A boundary line between the first and second portions extends along a direction from the coupling area toward the active area and intersects the first wiring line.

### CROSS-REFERENCE TO RELATED APPLICATION

This application claims the benefit of priority from Japanese Patent Application No. 2023-066374 filed on Apr. 14, 2023 and International Patent Application No. PCT/JP2024/013415 filed on Apr. 1, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (refer to, for example, Japanese Patent Application Laid-open Publication No. 2009-032005). Among such optical sensors, sensors are known each including a plurality of photodiodes in which an organic semiconductor material is used as an active layer. The organic semiconductor material is disposed between lower and upper electrodes, and signal lines are electrically coupled to the lower electrodes of the photodiodes to output detection signals to a detection circuit.

When a ring-shaped housing is bent toward the vertical direction, stress is concentrated on a step provided between a sensor substrate and a protective layer provided on top of the sensor substrate because the protective layer has a smaller area than the sensor substrate therebelow. As a result, wiring cracks may occur.

For the foregoing reasons, there is a need for a detection device that can ease stress concentration on wiring at a step and reduce wiring cracks.

### SUMMARY

According to an aspect, a detection device includes: a photodiode including a lower electrode, a lower buffer layer, an active layer, an upper buffer layer, and an upper electrode; a first substrate; a first insulating layer located between the first substrate and the photodiode; a second substrate that covers at least the photodiode so as to sandwich the photodiode between the first substrate and the second substrate, and has an area smaller than the first substrate; a second insulating layer located between the second substrate and the photodiode; an active area including the active layer of the photodiode; a coupling area in which a coupling part provided at an end of the first substrate is located; a peripheral area between the active area and the coupling area; a sealing film that seals the active area and the peripheral area; and a first wiring line that couples the lower electrode to the coupling part. The peripheral area includes: a first portion that includes the first insulating layer, the sealing film, the second insulating layer, and the second substrate on the first substrate; and a second portion in which at least one of the sealing film, the second insulating layer, and the second substrate is removed compared with the first portion. A boundary line between the first portion and the second portion extends along a first direction from the coupling area toward the active area. The boundary line intersects the first wiring line.

According to another aspect, a detection device includes: a photodiode including a lower electrode, a lower buffer layer, an active layer, an upper buffer layer, and an upper electrode; a first substrate; a first insulating layer located between the first substrate and the photodiode; a second substrate that covers at least the photodiode so as to sandwich the photodiode between the first substrate and the second substrate, and has an area smaller than the first substrate; a second insulating layer located between the second substrate and the photodiode; an active area including the active layer of the photodiode; a coupling area in which a coupling part provided at an end of the first substrate is located; a peripheral area between the active area and the coupling area; a sealing film that seals the active area and the peripheral area; and a first wiring line that couples the lower electrode to the coupling part. The peripheral area includes: a first portion that comprises the first insulating layer, the sealing film, the second insulating layer, and the second substrate on the first substrate; and a second portion including the first insulating layer on the first substrate. A boundary line between the first portion and the second portion intersects the first wiring lines along a second direction intersecting the first direction from the coupling area toward the active area. A flexible printed circuit board is coupled to the coupling area. A protective film covers the second substrate so as to overlap an entire surface of the active area and extends to the peripheral area. A portion of the flexible printed circuit board is sandwiched between the protective film and the first substrate.

### BRIEF DESCRIPTION OF THE DRAWINGS

FIG. 1 is a schematic view illustrating an exemplary external appearance when a state of a finger accommodated inside a detection device according to a first embodiment is viewed from a lateral side of a housing;

FIG. 2 is a schematic sectional view taken along section II-II′ illustrated in FIG. 1;

FIG. 3 is a development view illustrating an exemplary development of optical sensors of the detection device illustrated in FIG. 1;

FIG. 4 is a configuration diagram illustrating an exemplary configuration of a first optical sensor and a second optical sensor illustrated in FIG. 3;

FIG. 5 is a schematic sectional view illustrating an exemplary multilayer configuration of the optical sensor taken along section V-V′ illustrated in FIG. 4;

FIG. 6 is a schematic sectional view illustrating an exemplary multilayer configuration of the optical sensor taken along section VI-VI′ illustrated in FIG. 4;

FIG. 7 is a schematic sectional view illustrating an exemplary multilayer configuration of the optical sensor taken along section VII-VII′ illustrated in FIG. 4;

FIG. 8 is a schematic sectional view illustrating an exemplary multilayer configuration of the optical sensor taken along section VIII-VIII′ illustrated in FIG. 4;

FIG. 9 is a configuration diagram illustrating an exemplary configuration of the first optical sensor and the second optical sensor according to a comparative example;

FIG. 10 is a schematic sectional view illustrating an exemplary multilayer configuration of the optical sensor illustrated in FIG. 9;

FIG. 11 is a schematic sectional view illustrating a form of the optical sensor illustrated in FIG. 9 when being bent toward a third direction;

FIG. 12 is a configuration diagram illustrating an exemplary configuration of the first optical sensor and the second optical sensor according to a second embodiment;

FIG. 13 is a schematic sectional view illustrating an exemplary multilayer configuration of the optical sensor taken along section XIII-XIII′ illustrated in FIG. 12;

FIG. 14 is a schematic sectional view illustrating an exemplary multilayer configuration of the optical sensor according to a third embodiment;

FIG. 15 is a configuration diagram illustrating an exemplary configuration of the first optical sensor and the second optical sensor according to a fourth embodiment;

FIG. 16 is a configuration diagram illustrating an exemplary configuration of the first optical sensor and the second optical sensor according to a fifth embodiment; and

FIG. 17 is a schematic sectional view illustrating an exemplary multilayer configuration of the optical sensor illustrated in FIG. 16.

### DETAILED DESCRIPTION

The following describes modes (embodiments) for carrying out the disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

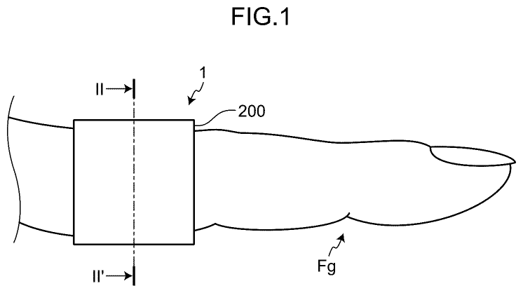

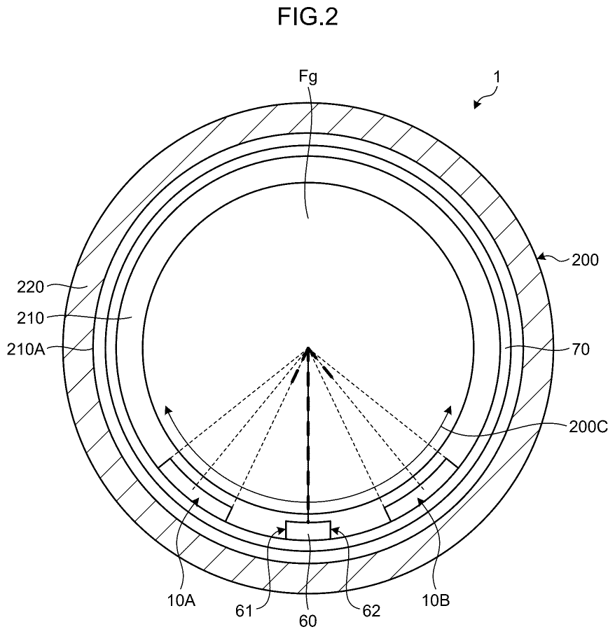

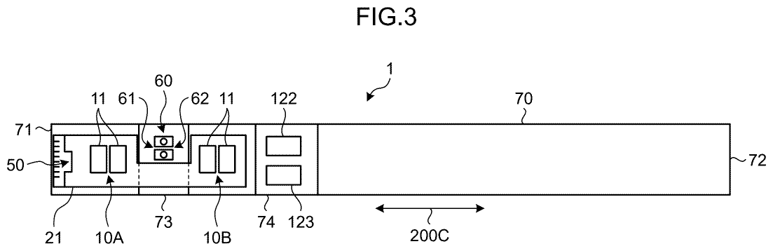

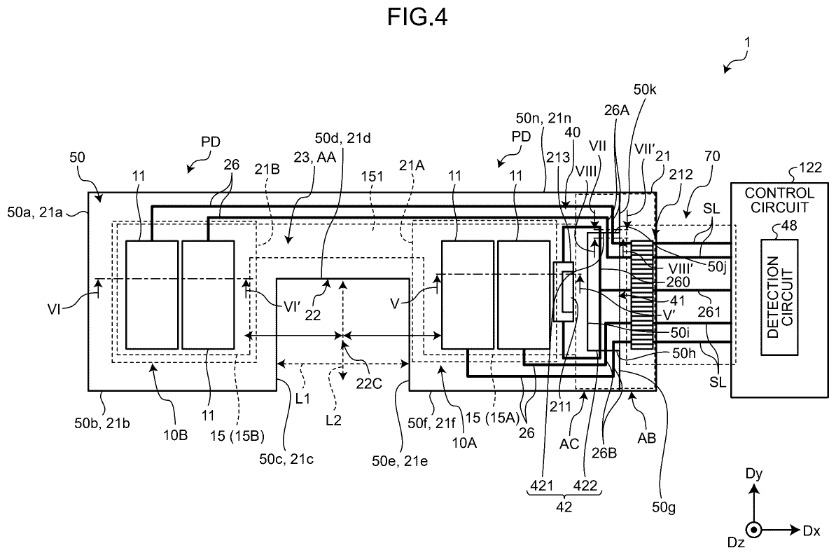

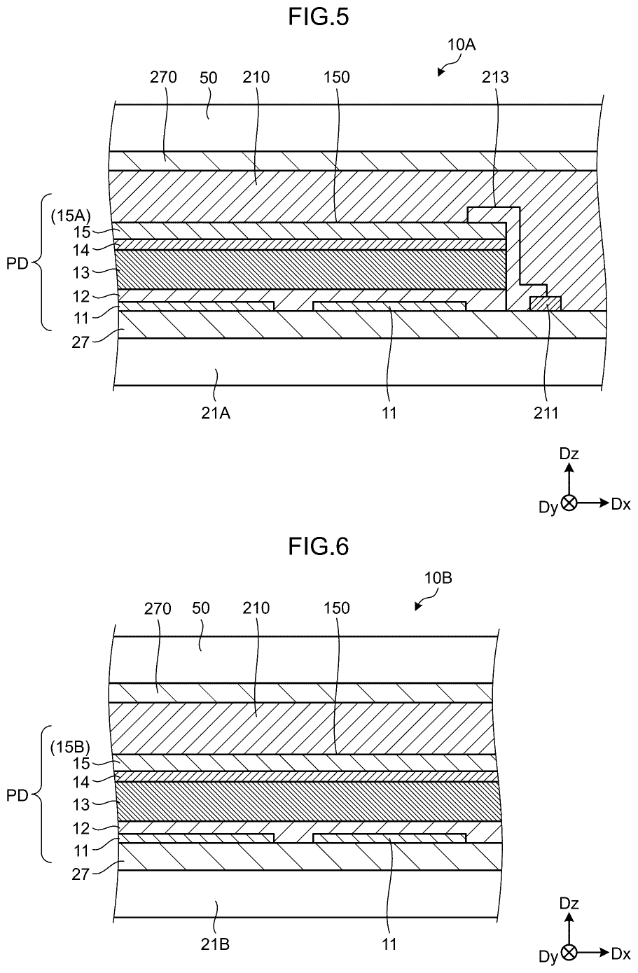

FIG. 1 is a schematic view illustrating an exemplary external appearance when a state of a finger accommodated inside a detection device according to a first embodiment is viewed from a lateral side of a housing. FIG. 2 is a schematic sectional view taken along section II-II′ illustrated in FIG. 1. FIG. 3 is a development view illustrating an exemplary development of optical sensors of the detection device illustrated in FIG. 1. FIG. 4 is a configuration diagram illustrating an exemplary configuration of a first optical sensor and a second optical sensor illustrated in FIG. 3. FIG. 5 is a schematic sectional view illustrating an exemplary multilayer configuration of the optical sensor taken along section V-V′ illustrated in FIG. 4. FIG. 6 is a schematic sectional view illustrating an exemplary multilayer configuration of the optical sensor taken along section VI-VI′ illustrated in FIG. 4.

A detection device 1 illustrated in FIG. 1 is a finger ring-shaped device that can be worn on and removed from a human body and is worn on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body, and is an object to be measured. The detection device 1 is formed as a finger ring or a wristband so as to be easily carried by a user. In the following description, the detection device 1 is assumed to be used as a finger ring.

As illustrated in FIG. 2, the detection device 1 includes a housing 200, a light source 60, a first optical sensor 10A, and a second optical sensor 10B. The detection device 1 is a device that includes a battery (not illustrated) in the housing 200 and is operated by power from the battery.

The housing 200 is formed in a ring shape (annular shape) that can be worn on the finger Fg, and is a wearable member to be worn on the living body. In the example illustrated in FIG. 2, the housing 200 includes a sealing film 210 and an exterior member 220. In the housing 200, the sealing film 210 is integrated with the exterior member 220 to be formed into a ring shape. The sealing film 210 accommodates therein the light source 60, the first optical sensor 10A, the second optical sensor 10B, and so forth. The sealing film 210 is formed of a housing material, such as a light-transmitting synthetic resin or silicon, into a ring shape. The exterior member 220 has a surface of the housing 200 that covers an outer peripheral surface 210A of the sealing film 210. The exterior member 220 is formed, for example, of a member of a metal or a non-light-transmitting synthetic resin, into a ring shape. The housing 200 accommodates, in the sealing film 210, a flexible printed circuit board 70 on which the light source 60, the first optical sensor 10A, the second optical sensor 10B, and so forth are mounted. The flexible printed circuit board 70 is accommodated in the housing 200, for example, by forming the housing 200 by filling the periphery of the flexible printed circuit board 70 formed into a ring shape with a filling member in a mold.

As illustrated in FIG. 3, the flexible printed circuit board 70 is formed into a deformable band shape, and is formed into a ring shape by coupling one end 71 to the other end 72. The flexible printed circuit board 70 has a first mounting area 73 and a second mounting area 74. The first mounting area 73 is an area where the light source 60 and so forth are mounted. The second mounting area 74 is an area where a control circuit 122, a power supply circuit 123, and so forth are mounted. A first substrate 21 is mounted on the flexible printed circuit board 70 so as to straddle the vicinity of the light source 60 in the first mounting area 73. A second substrate 50 is provided on the first substrate 21. The flexible printed circuit board 70 electrically couples the light source 60, the first optical sensor 10A, the second optical sensor 10B, and so forth to the control circuit 122.

In the present embodiment, the first and the second optical sensors 10A and 10B are provided so as to interpose the light source 60 therebetween in a circumferential direction 200C. That is, in the detection device 1, the first optical sensor 10A, the light source 60, and the second optical sensor 10B are arranged in this order in the circumferential direction 200C. The first and the second optical sensors 10A and 10B are arranged so as to interpose the light source 60 therebetween in the circumferential direction 200C. Thereby, light emitted by the light source 60 can be detected over a wide area of the housing 200.

The first substrate 21 is an insulating substrate, is formed, for example, of polyethylene terephthalate (PET) that is a film-like synthetic resin, and formed into a band shape. The first substrate 21 is a deformable substrate on which the first and the second optical sensors 10A and 10B are mounted. The first substrate 21 can be curved toward a third direction Dz. When the sensor substrate 21 is mounted on the flexible printed circuit board 70, the first and the second optical sensors 10A and 10B are positioned on opposite sides of the light source 60 in the circumferential direction 200C of the housing 200. The first substrate 21 has a first area 21A where the first optical sensor 10A is mounted, and a second area 21B where the second optical sensor 10B is mounted. The first substrate 21 is formed as one substrate having the first area 21A and the second area 21B.

As with the first substrate 21, the second substrate 50 is an insulating substrate and is formed into a band shape composed, for example, of polyethylene terephthalate (PET) that is a film-like synthetic resin. The second substrate 50 covers the sealing film 210 and is a deformable substrate. The second substrate 50 can be curved toward the third direction Dz.

In the present embodiment, as illustrated in FIG. 2, the flexible printed circuit board 70 is accommodated in the housing 200 such that a surface provided with the first optical sensor 10A, the second optical sensor 10B, and the light source 60 faces an inner peripheral surface 200B of the housing 200. When the flexible printed circuit board 70 has a light-transmitting property, the first optical sensor 10A, the second optical sensor 10B, and the light source 60 may be mounted on the back surface opposite the front surface. In this case, the light source 60 only needs to be disposed so as to emit the light toward the flexible printed circuit board 70 and so that the light transmitted through the flexible printed circuit board 70 is emitted toward outside the housing 200.

As illustrated in FIG. 2, the light source 60 is provided in the sealing film 210 of the housing 200, and is configured to be capable of emitting light toward an object to be detected such as the finger Fg wearing the ring-shaped housing 200. For example, inorganic light-emitting diodes (LEDs) or organic electroluminescent (EL) diodes (organic light-emitting diodes (OLEDs)) are used as the light source 60. The light source 60 emits light having predetermined wavelengths. In the present embodiment, the light source 60 includes a plurality of light sources so as to be capable of emitting near-infrared light, red light, and green light.

The light emitted from the light source 60 is reflected by a surface of the object to be detected, such as the finger Fg, and enters the first and the second optical sensors 10A and 10B. Thereby, the detection device 1 can detect a fingerprint by detecting a shape of asperities on a surface of the finger Fg or the like. Alternatively, the light emitted from the light source 60 may be reflected in the finger Fg or the like, or transmitted through the finger Fg or the like and enter the first and the second optical sensors 10A and 10B. Thereby, the detection device 1 can detect the information on the living body in the finger Fg or the like. Examples of the information on the living body include, but are not limited to, pulse waves, pulsation, and a vascular image of the finger or a palm. That is, the detection device 1 may be configured as a fingerprint detection device that detects the fingerprint or a vein detection device that detects a pattern of blood vessels such as veins.

Each of the first and the second optical sensors 10A and 10B detects light emitted by the light source 60 and reflected by the finger Fg or the like, light directly incident on the optical sensor, and other light. The first and the second optical sensors 10A and 10B are each an organic photodiode (OPD). The first optical sensor 10A is provided in the housing 200 so as to be adjacent to one end 61 of the light source 60 in the circumferential direction 200C of the housing 200. The second optical sensor 10B is provided in the housing 200 so as to be adjacent to another end 62 of the light source 60 in the circumferential direction 200C of the housing 200.

As illustrated in FIG. 3, the first and the second optical sensors 10A and 10B each include a photodiode PD (refer to FIG. 4) that is an organic photodiode. Each of the first and the second optical sensors 10A and 10B has a configuration with two lower electrodes 11 arranged along the circumferential direction 200C. The first and the second optical sensors 10A and 10B are mounted on one first substrate 21 and are electrically coupled to the flexible printed circuit board 70 via the first substrate 21. The first substrate 21 has a notch 22 (refer to FIG. 4) between the first and the second optical sensors 10A and 10B in the circumferential direction 200C of the housing 200.

In the following description, a first direction Dx is one direction in a plane parallel to the first substrate 21 and is the same direction as the circumferential direction 200C. A second direction Dy is one direction in the plane parallel to the first substrate 21 and is a direction orthogonal to the first direction Dx. The second direction Dy may non-orthogonally intersect the first direction Dx. The third direction Dz is a direction orthogonal to the first direction Dx and the second direction Dy. The third direction Dz is a direction normal to the first substrate 21. The term “plan view” refers to a positional relation when viewed along a direction orthogonal to the first substrate 21.

As illustrated in FIG. 4, the first optical sensor 10A has a configuration in which the two lower electrodes 11 arranged in the first direction Dx and one upper electrode 15A are stacked together. The second optical sensor 10B has a configuration in which the two lower electrodes 11 arranged in the first direction Dx and one upper electrode 15B are stacked together. An upper electrode 15 includes the upper electrode 15A of the first optical sensor 10A and the upper electrode 15B of the second optical sensor 10B. Each of the upper electrodes 15A and 15B covers the two lower electrodes 11 in plan view. The upper electrodes 15A and 15B are electrically coupled to each other by an electrode connector 151. The upper electrodes 15A and 15B and the electrode connector 151 are integrally formed.

The first substrate 21 includes a power supply electrode 211 that extends along the second direction Dy. The power supply electrode 211 is electrically coupled to a coupling part 212 (terminal) of the first substrate 21 through a conductor 213, and is supplied with a sensor power supply signal (sensor power supply voltage) from the power supply circuit 123 (refer to FIG. 3) via the coupling part 212. The upper electrode 15 is electrically coupled to the power supply electrode 211 by the conductor 213. The conductor 213 is provided on the first substrate 21 so as to extend overlapping both the upper electrode 15 and the power supply electrode 211, and is formed of a conductive material. With this configuration, the upper electrode 15 is supplied with the sensor power supply signal from the power supply circuit 123 via the power supply electrode 211.

A plurality of first wiring lines 26 on the first substrate 21 are coupled to a detection circuit 48 included in the control circuit 122 via a plurality of signal lines SL of the flexible printed circuit board 70. The detection circuit 48 is electrically coupled to the lower electrodes 11 of the first and the second optical sensors 10A and 10B via the signal lines SL. The detection circuit 48 may be formed as a circuit separate from the control circuit 122.

The first wiring lines 26 are formed, for example, of metal wiring, and is formed of a material having better conductivity than the lower electrodes 11 of the photodiode PD. The first wiring lines 26 are formed of a light-transmitting conductive material such as indium tin oxide (ITO). The first wiring lines 26 are provided in a layer between the first substrate 21 and the photodiode PD in the third direction Dz. The first wiring lines 26 are electrically coupled to the lower electrodes 11 and the coupling part 212 of the first substrate 21. The first wiring lines 26 may be formed, for example, in the same layer as the lower electrodes 11, or formed of a metal.

A second wiring line 260 is electrically coupled to the power supply electrode 211 by the conductor 213.

The second wiring line 260 is formed, for example, of metal wiring, and is formed of a conductive material. The second wiring line 260 is formed of a material having better conductivity than the upper electrode 15. The second wiring line 260 is provided in a layer between the first substrate 21 and the photodiode PD in the third direction Dz. The second wiring line 260 is electrically coupled to the upper electrode 15 and the coupling part 212. The second wiring line 260 may be formed, for example, in the same layer as the upper electrode 15, or formed of a metal. The second wiring line 260 may be a shield layer.

The control circuit 122 is a circuit that controls detection operations by supplying control signals to a plurality of the photodiodes PD. Each of the photodiodes PD outputs an electrical signal in response to the light emitted thereto as a detection signal Vdet to the detection circuit 48. The second wiring line 260 is coupled to the control circuit 122 via wiring 261 that supplies a power supply voltage to the second wiring line 260. In the present embodiment, the detection signals Vdet of the photodiodes PD are sequentially output to the detection circuit 48 in a time-division manner. In other words, the signal lines SL are sequentially electrically coupled to the detection circuit 48 in a time-division manner. Thereby, the detection device 1 detects information on the object to be detected, based on the detection signals Vdet from the photodiodes PD.

The first substrate 21 has a first side surface 21a, a second side surface 21b, a third side surface 21c, a fourth side surface 21d, a fifth side surface 21e, a sixth side surface 21f, and a seventh side surface 21n.

The second substrate 50 has a first side surface 50a, a second side surface 50b, a third side surface 50c, a fourth side surface 50d, a fifth side surface 50e, a sixth side surface 50f, a seventh side surface 50g, an eighth side surface 50h, a ninth side surface 50i, a tenth side surface 50j, an eleventh side surface 50k, and a twelfth side surface 50n.

The first side surface 21a and the first side surface 50a are planar, parallel to each other, equal in length, and overlap each other. The second side surface 21b and the second side surface 50b are planar, parallel to each other, equal in length, and overlap each other. The third side surface 21c and the third side surface 50c are planar, parallel to each other, equal in length, and overlap each other. The fourth side surface 21d and the fourth side surface 50d are planar, parallel to each other, equal in length, and overlap each other. The fifth side surface 21e and the fifth side surface 50e are planar, parallel to each other, equal in length, and overlap each other.

The sixth side surface 50f is parallel to the sixth side surface 21f, but the sixth side surface 50f is smaller in length than the sixth side surface 21f. The twelfth side surface 50n is parallel to the seventh side surface 21n, but the twelfth side surface 50n is smaller in length than the seventh side surface 21n. The first substrate 21 has no sides at portions overlapping the seventh side surface 50g, the eighth side surface 50h, the ninth side surface 50i, the tenth side surface 50j, and the eleventh side surface 50k. Thus, the first substrate 21 is provided thereon with the second substrate 50 that covers the photodiodes PD and has a smaller area than the first substrate 21.

As illustrated in FIG. 5, the first optical sensor 10A includes the first substrate 21 (first area 21A), the photodiodes PD, and the second substrate 50 that faces the first substrate 21. In the present embodiment, the first optical sensor 10A further includes a first insulating layer 27 and a second insulating layer 270.

The first insulating layer 27 is provided on the upper side of the first substrate 21. The first insulating layer 27 is located between the first substrate 21 and the photodiode PD. The second insulating layer 270 is provided on the upper side of the photodiode PD. The second insulating layer 270 is located between the second substrate 50 and the photodiode PD. The first insulating layer 27 and the second insulating layer 270 may be inorganic insulating films or organic insulating films.

The photodiode PD is provided on the upper side of the first insulating layer 27. The photodiode PD includes the lower electrodes 11, a lower buffer layer 12, an active layer 13, an upper buffer layer 14, and the upper electrode 15 (15A). In the photodiode PD, the lower electrodes 11, the lower buffer layer 12 (hole transport layer), the active layer 13, the upper buffer layer 14 (electron transport layer), and the upper electrode 15 are stacked in this order in the third direction Dz orthogonal to the first substrate 21.

Each of the lower electrodes 11 is an anode electrode of the photodiode PD and is formed of a light-transmitting conductive material such as indium tin oxide (ITO), for example. The active layer 13 changes in characteristics (such as voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly (3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 is a hole transport layer. The upper buffer layer 14 is an electron transport layer. The lower buffer layer 12 and the upper buffer layer 14 are provided to facilitate holes and electrons generated in the active layer 13 to reach the lower electrodes 11 or the upper electrode 15. The lower buffer layer 12 (hole transport layer) is in direct contact with the tops of the lower electrodes 11 and is also provided in an area between the adjacent lower electrodes 11. The active layer 13 is in direct contact with the top of the lower buffer layer 12. The material of the hole transport layer is a metal oxide layer. Tungsten oxide (WO₃), molybdenum oxide, or the like is used as the metal oxide layer.

The upper buffer layer 14 (electron transport layer) is in direct contact with the top of the active layer 13, and the upper electrode 15 is in direct contact with the top of the upper buffer layer 14. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is a cathode electrode of the photodiode PD, and is continuously formed over the entire first and second optical sensors 10A and 10B. In other words, the upper electrode 15 is continuously provided on the photodiodes PD. The upper electrode 15 faces the lower electrodes 11 with the lower buffer layer 12, the active layer 13, and the upper buffer layer 14 interposed therebetween. The upper electrode 15 is formed, for example, of a light-transmitting conductive material such as ITO or indium zinc oxide (IZO). A portion of an edge region of an upper surface 150 of the upper electrode 15 is electrically coupled to the conductor 213. In the first optical sensor 10A, the photodiode PD is well sealed by providing the sealing film 210 on the upper electrode 15 and so forth.

As illustrated in FIG. 6, the second optical sensor 10B includes the lower electrodes 11 of the second optical sensor 10B in the second area 21B of the first substrate 21 different from the area for the lower electrodes 11 of the first optical sensor 10A. The lower electrodes 11 are covered with the lower buffer layer 12, the active layer 13, the upper buffer layer 14, and the upper electrode 15 (15B). In the present embodiment, the second optical sensor 10B includes the first substrate 21 (second area 21B), the photodiode PD, the first insulating layer 27, the second substrate 50 that faces the first substrate 21, and the second insulating layer 270. The photodiode PD and the first insulating layer 27 of the second optical sensor 10B have the same configurations as those of the photodiode PD and the first insulating layer 27 of the first optical sensor 10A described above. That is, the photodiode PD of the second optical sensor 10B includes the lower electrodes 11, the lower buffer layer 12, the active layer 13, the upper buffer layer 14, and the upper electrode 15 (15B). In the present embodiment, the photodiodes PD of the first and the second optical sensors 10A and 10B are organic photodiodes.

As illustrated in FIG. 4, the first substrate 21 has the first area 21A for the first optical sensor 10A and the second area 21B for the second optical sensor 10B, and is integrally formed into one common substrate. The first substrate 21 has the notch 22 formed between the first area 21A for the first optical sensor 10A and the second area 21B for the second optical sensor 10B in the first direction Dx. The first substrate 21 has the notch 22 between the first and the second optical sensors 10A and 10B, and a joint 23 that is adjacent to the notch 22 and positioned between the first and the second optical sensors 10A and 10B.

The notch 22 is formed to have a length L1 longer than the length of the light source 60 in the first direction Dx. The notch 22 is formed to have a length L2 longer than the length of the light source 60 and shorter than the length (width) of the first substrate 21 in the second direction Dy. The notch 22 is formed such that the distance between a center 22C and one side of the lower electrode 11 of the first optical sensor 10A is equal to the distance between the center 22C and one side of the lower electrode 11 of the second optical sensor 10B in the first direction Dx. The first substrate 21 is integrally formed by connecting the first optical sensor 10A to the second optical sensor 10B via the joint 23 adjacent to the notch 22. The lower buffer layer 12, the active layer 13, the upper buffer layer 14, and the electrode connector 151 of the upper electrodes 15 are arranged at the joint 23. With this configuration, the joint 23 integrally forms the upper electrodes 15 of the first optical sensor 10A and the second optical sensor 10B. The notch 22 is formed in a shape that allows the light source 60 to be located therein. In the present embodiment, the notch 22 is formed into a substantially rectangular shape in plan view, but may have a semicircular, triangular, polygonal, or other shape, for example. The electrode connector 151 is provided on the joint 23 of the first substrate 21 so as to be stacked on top of the upper buffer layer 14, the active layer 13, and the lower buffer layer 12.

An area where the active layer 13 of the photodiode PD (refer to FIG. 5) is located is defined as an active area AA. An area on the coupling part 212 side of a plane connecting the seventh side surface 50g to the tenth side surface 50j is defined as a coupling area AB. An area between the active area AA and the coupling area AB is defined as a peripheral area AC. The one end 71 (refer to FIG. 3) of the flexible printed circuit board 70 including the signal lines SL is electrically coupled to the coupling area AB by an anisotropic conductive resin or the like.

The peripheral area AC has a first portion 40 and a second portion 41. The first portion 40 includes the first insulating layer 27, the sealing film 210, the second insulating layer 270, and the second substrate 50 on the first substrate 21. The second portion 41 is a portion where at least one of the sealing film 210, the second insulating layer 270, and the second substrate 50 is removed compared with the first portion 40. The sealing film 210 seals the active area AA and the peripheral area AC.

A boundary line 42 between the first portion 40 and the second portion 41 along a direction from the coupling area AB toward the active area AA is parallel to the first direction Dx, and a direction in which the first wiring lines 26 intersecting the boundary line 42 extend is orthogonal to the first direction Dx. The boundary line 42 has a first side 421 and a second side 422 opposite the first side 421 in the second direction Dy. The first side 421 is located on the tenth side surface 50j. The second side 422 is located on the eighth side surface 50h. The first wiring lines 26 includes wiring lines 26A and wiring lines 26B. The wiring lines 26A intersect the first side 421, and the wiring lines 26B intersect the second side 422. The second wiring line 260 intersects the first side 421 and the second side 422.

A step corresponding to the thickness of the second substrate 50 is formed in the third direction on each of the seventh side surface 50g, the ninth side surface 50i, and the eleventh side surface 50k, but the first wiring lines 26 and the second wiring line 260 do not intersect these side surfaces. Therefore, when the detection device 1 is bent toward the third direction, stress generated at the step is difficult to be applied to the first wiring lines 26 and the second wiring line 260.

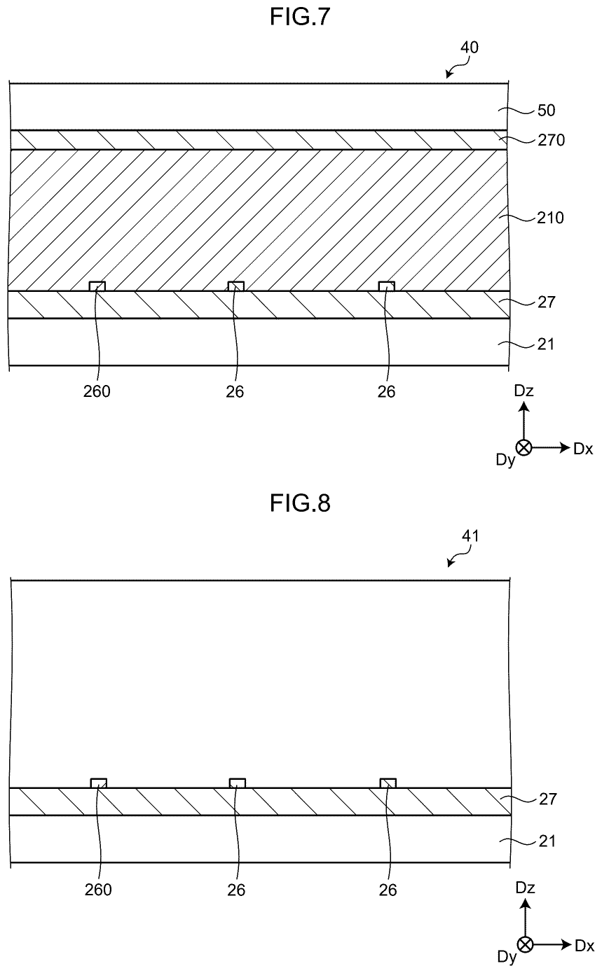

FIG. 7 is a schematic sectional view illustrating an exemplary multilayer configuration of the optical sensor taken along section VII-VII′ illustrated in FIG. 4. FIG. 8 is a schematic sectional view illustrating an exemplary multilayer configuration of the optical sensor taken along section VIII-VIII′ illustrated in FIG. 4.

As illustrated in FIG. 7, in the first portion 40, the first substrate 21, the first insulating layer 27, the sealing film 210, the first insulating layer 27, and the second substrate 50 are stacked in this order. The first insulating layer 27 is provided on the upper surface of the first substrate 21, and the first wiring lines 26 and the second wiring line 260 are provided on the upper surface of first insulating layer 27. The lower electrodes 11 are arranged on the upper surface of the first insulating layer 27 (refer to FIGS. 5 and 6).

As illustrated in FIG. 8, the second portion 41 includes the first substrate 21, the first insulating layer 27, the first wiring lines 26, and the second wiring line 260. The first wiring lines 26 and the second wiring line 260 are provided on the upper surface of the first insulating layer 27. The second portion 41 includes the first insulating layer 27 on the first substrate 21, with the sealing film 210, the second insulating layer 270, and the second substrate 50 removed.

The exemplary configuration of the detection device 1 according to the present embodiment has been described above. The configuration described above using FIGS. 1 to 8 is merely an example, and the configuration of the detection device 1 according to the present embodiment is not limited to the example. The configuration of the detection device 1 according to the present embodiment can be flexibly modified according to requirements or operations.

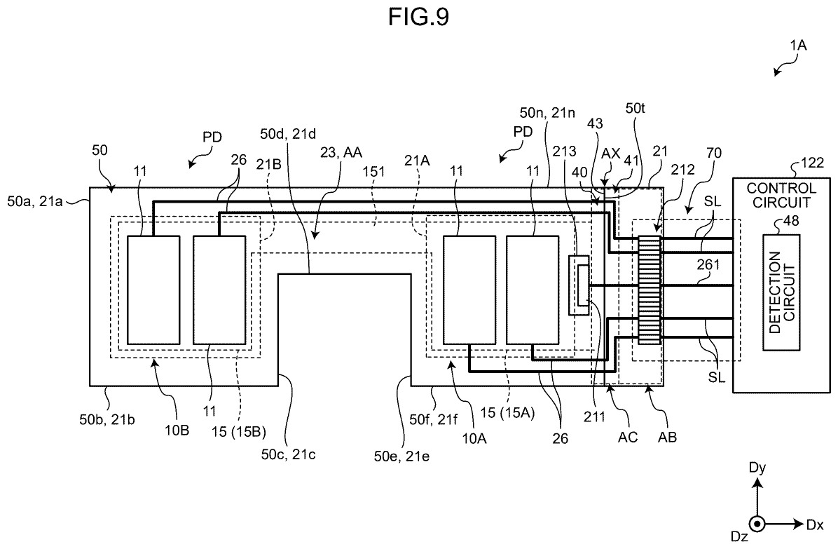

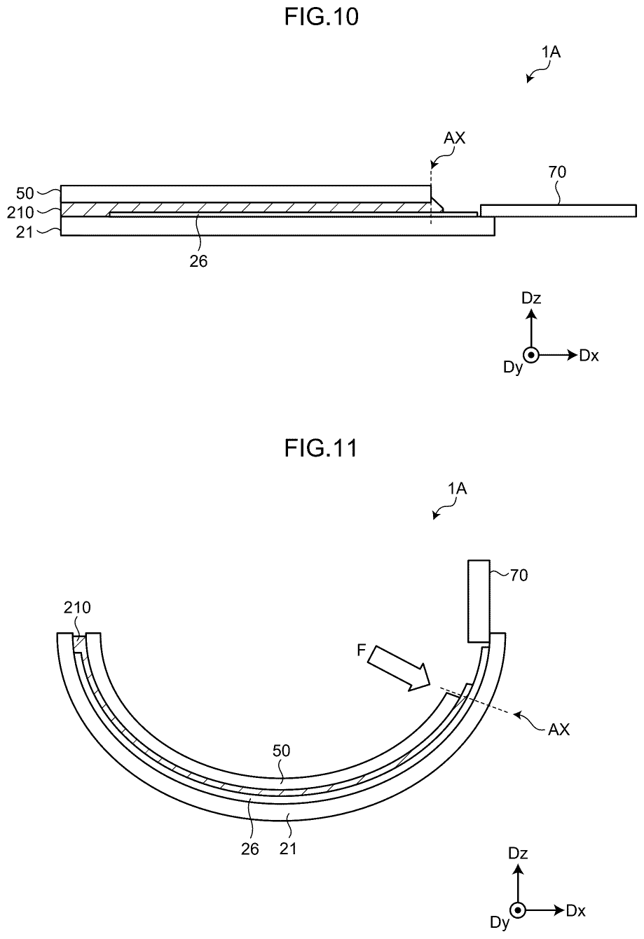

FIG. 9 is a configuration diagram illustrating an exemplary configuration of the first optical sensor and the second optical sensor according to a comparative example. FIG. 10 is a schematic sectional view illustrating an exemplary multilayer configuration of the optical sensor illustrated in FIG. 9. FIG. 11 is a schematic sectional view illustrating a form of the optical sensor illustrated in FIG. 9 when being bent toward the third direction.

As illustrated in FIG. 9, the second substrate 50 has the first side surface 50a, the second side surface 50b, the third side surface 50c, the fourth side surface 50d, the fifth side surface 50e, the sixth side surface 50f, the twelfth side surface 50n, and a thirteenth side surface 50t.

In a detection device 1A according to the comparative example, the first side surface 21a and the first side surface 50a are planar, parallel to each other, equal in length and, overlap each other. The second side surface 21b and the second side surface 50b are planar, parallel to each other, equal in length, and overlap each other. The third side surface 21c and the third side surface 50c are planar, parallel to each other, equal in length, and overlap each other. The fourth side surface 21d and the fourth side surface 50d are planar, parallel to each other, equal in length, and overlap each other. The fifth side surface 21e and the fifth side surface 50e are planar, parallel to each other, equal in length, and overlap each other.

The sixth side surface 50f is parallel to the sixth side surface 21f, but the sixth side surface 50f is smaller in length than the sixth side surface 21f. The twelfth side surface 50n is parallel to the seventh side surface 21n, but the twelfth side surface 50n is smaller in length than the seventh side surface 21n. The first substrate 21 has no side at a portion overlapping the thirteenth side surface 50t. Thus, the first substrate 21 is provided thereon with the second substrate 50 that covers the photodiodes PD and has a smaller area than the first substrate 21.

As illustrated in FIG. 9, a boundary line 43 is provided between the first portion 40 and the second portion 41 on the thirteenth side surface 50t. The first wiring lines 26 intersect the boundary line 43.

As illustrated in FIG. 10, the area of the second substrate 50 on the upper side is smaller than that of the first substrate 21 on the lower side. As a result, a step AX corresponding to the thickness of the second substrate 50 is formed in the third direction of the thirteenth side surface 50t on the flexible printed circuit board 70 side.

Therefore, as illustrated in FIG. 11, when the detection device 1A is curved toward the third direction, an end on the flexible printed circuit board 70 side of the first substrate 21 deflects in a direction in which a force is applied. An area provided with only the first substrate 21 and an area where the second substrate 50 and the first substrate 21 overlap are different in thickness and thus, different in amount of deflection even when the same bending force is applied. Due to this difference in amount of deflection, an end on the flexible printed circuit board 70 side of the second substrate 50 is pressed against a surface of the first substrate 21, and stress F is concentrated on the step AX. As a result, cracks may occur in the first wiring lines 26 that intersect the step AX.

In contrast, in the detection device 1 according to the first embodiment, the first wiring lines 26 and the second wiring line 260 intersect none of the seventh side surface 50g, the ninth side surface 50i, and the eleventh side surface 50k, where a step corresponding to the thickness of the first substrate 21 is formed. In contrast to this, the first wiring lines 26 and the second wiring line 260 intersect the first and the second sides 421 and 422. However, when the detection device 1 is bent toward the third direction, the first wiring lines 26 and the second wiring line 260 are less subject to stress on the first and the second sides 421 and 422. As a result, cracks in the first wiring lines 26 and the second wiring line 260 that intersect the first and the second sides 421 and 422 are reduced.

Since the second substrate 50, the second insulating layer 270, and the sealing film 210 are not provided in the second portion 41, the first wiring lines 26 are exposed, and when the detection device 1 is bent toward the third direction, the stress applied to the second portion 41 is eased and the stress becomes difficult to be applied to the first wiring lines 26.

### Second Embodiment

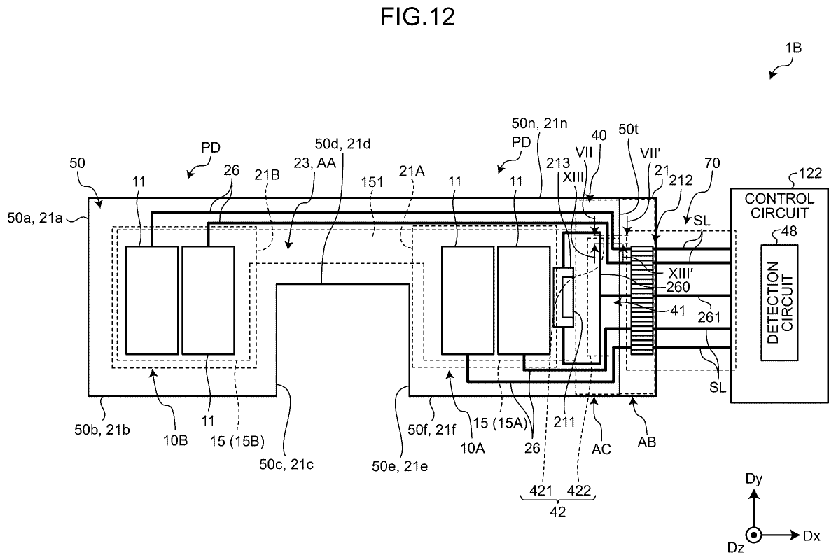

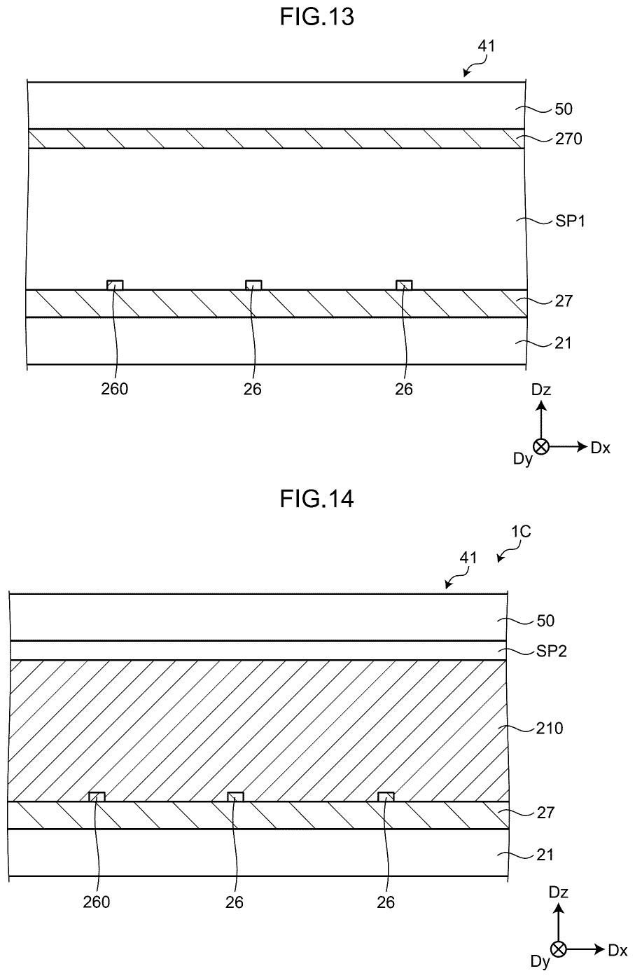

FIG. 12 is a configuration diagram illustrating an exemplary configuration of the first optical sensor and the second optical sensor according to a second embodiment. FIG. 13 is a schematic sectional view illustrating an exemplary multilayer configuration of the optical sensor taken along section XIII-XIII′ illustrated in FIG. 12. In the following description, the same components as those described in the embodiment described above are denoted by the same reference numerals, and will not be described again. The description of the first portion 40 is the same as that of the first embodiment and is therefore omitted. As illustrated in FIG. 12, the second substrate 50 has the first side surface 50a, the second side surface 50b, the third side surface 50c, the fourth side surface 50d, the fifth side surface 50e, the sixth side surface 50f, the twelfth side surface 50n, and the thirteenth side surface 50t.

In a detection device 1B according to the second embodiment, the first side surface 21a and the first side surface 50a are planar, parallel to each other, equal in length, and overlap each other. The second side surface 21b and the second side surface 50b are planar, parallel to each other, equal in length, and overlap each other. The third side surface 21c and the third side surface 50c are planar, parallel to each other, equal in length, and overlap each other. The fourth side surface 21d and the fourth side surface 50d are planar, parallel to each other, equal in length, and overlap each other. The fifth side surface 21e and the fifth side surface 50e are planar, parallel to each other, equal in length, and overlap each other.

The sixth side surface 50f is parallel to the sixth side surface 21f, but the sixth side surface 50f is smaller in length than the sixth side surface 21f. The twelfth side surface 50n is parallel to the seventh side surface 21n, but the twelfth side surface 50n is smaller in length than the seventh side surface 21n. The thirteenth side surface 50t is located at a portion overlapping the first substrate 21 and overlap no side surface of the first substrate 21. Thus, the first substrate 21 is provided thereon with the second substrate 50 that covers the photodiode PD and has a smaller area than the first substrate 21.

As illustrated in FIG. 12, in the second portion 41, the first substrate 21 is covered with the second substrate 50. The first wiring lines 26 and the second wiring line 260 intersect the thirteenth side surface 50t that is a boundary between a portion with the second substrate 50 and a portion without the second substrate 50.

As illustrated in FIG. 13, the second portion 41 includes the first substrate 21, the first insulating layer 27, the first wiring lines 26, the second wiring line 260, the second insulating layer 270, and the second substrate 50. The first wiring lines 26 and the second wiring line are provided on the upper surface of the first insulating layer 27. In the second portion 41, the sealing film 210 is removed. A space SP1 is provided between the first insulating layer 27 and the second insulating layer 270. For example, an air layer is provided in the space SP1.

With this configuration, the sealing film 210 is not provided in the second portion 41. Therefore, the stress applied to the first side 421 and the second side 422 along the first direction Dx of the second portion 41 is eased, and the stress becomes difficult to be applied to the first wiring lines 26 and the second wiring line 260 that intersect the first side 421 and the second side 422. At intersections of the first wiring lines 26 and the second wiring line 260 with the thirteenth side surface 50t, the second portion 41 overlaps the thirteenth side surface 50t, and the sealing film 210 is not present. Therefore, even though the first wiring lines 26 and the second wiring line 260 intersect the thirteenth side surface 50t, the stress applied from the second substrate 50 to the first wiring lines 26 and the second wiring line 260 is eased.

### Third Embodiment

FIG. 14 is a schematic sectional view illustrating an exemplary multilayer configuration of the optical sensor according to a third embodiment. In the following description, the same components as those described in either of the embodiments described above are denoted by the same reference numerals, and will not be described again. The exemplary configuration of the first optical sensor and the second optical sensor according to the third embodiment is the same as that in the detection device according to the second embodiment, and therefore will not be described. The description of the first portion 40 is the same as that of the first embodiment and is therefore omitted.

As illustrated in FIG. 14, in the second portion 41 of a detection device 1C according to the third embodiment, the first substrate 21 is covered with the second substrate 50 in the same way as in the second embodiment.

As illustrated in FIG. 12, the first wiring lines 26 and the second wiring line 260 intersect the thirteenth side surface 50t that is the boundary between the portion with the second substrate 50 and the portion without the second substrate 50, in the same way as in the second embodiment.

As illustrated in FIG. 14, the second portion 41 includes the first substrate 21, the first insulating layer 27, the first wiring lines 26, the second wiring line 260, the sealing film 210, and the second substrate 50. The first wiring lines 26 and the second wiring line 260 are provided on the upper surface of the first insulating layer 27. In the second portion 41, the second insulating layer 270 is removed. A space SP2 is provided as a gap between the sealing film 210 and the second substrate 50. For example, an air layer is provided in the space SP2.

With this configuration, the second substrate 50 and the sealing film 210 are separated in the second portion 41. Therefore, the stress applied to the first side 421 and the second side 422 along the first direction Dx of the second portion 41 is eased, and the stress becomes difficult to be applied to the first wiring lines 26 and the second wiring line 260 that intersect the first side 421 and the second side 422. At intersections of the first wiring lines 26 and the second wiring line 260 with the thirteenth side surface 50t, the second portion 41 overlaps the thirteenth side surface 50t, and the second insulating layer 270 is not present. Therefore, even though the first wiring lines 26 and the second wiring line 260 intersect the thirteenth side surface 50t, the stress applied from the second substrate 50 to the first wiring lines 26 and the second wiring line 260 is eased.

### Fourth Embodiment

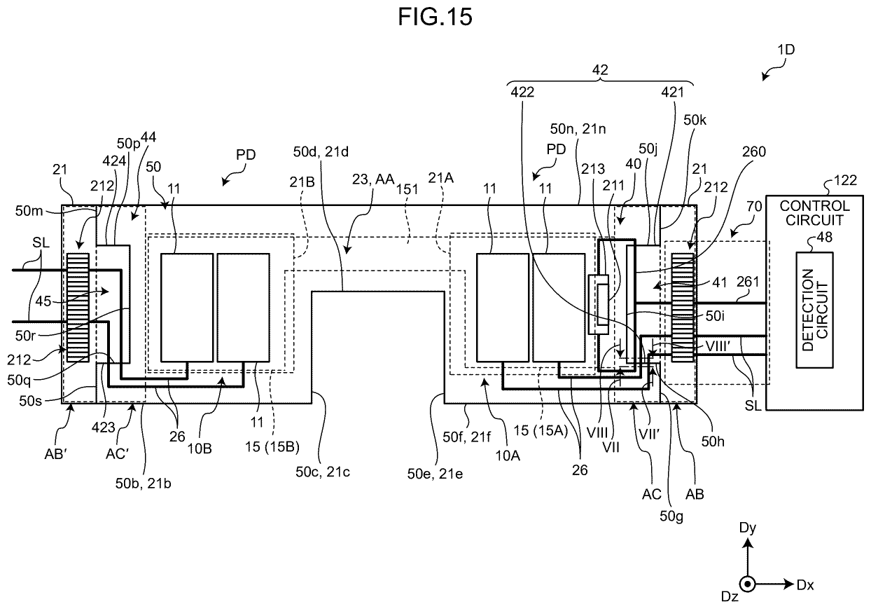

FIG. 15 is a configuration diagram illustrating an exemplary configuration of the first optical sensor and the second optical sensor according to a fourth embodiment. In the following description, the same components as those described in any one of the embodiments described above are denoted by the same reference numerals, and will not be described again. The description of the first portion 40 is the same as that of the first embodiment and is therefore omitted.

As illustrated in FIG. 15, a detection device 1D according to the fourth embodiment is provided with a coupling area AB′ including a coupling part 212 on the opposite side of the coupling area AB in the first direction Dx. The other end 72 of the flexible printed circuit board 70 (refer to FIG. 3) including the signal lines SL is electrically coupled to the coupling area AB′ by an anisotropic conductive resin or the like.

The second portion 41 includes the first substrate 21, the first insulating layer 27, the first wiring lines 26, and the second wiring line 260. The configuration of the second portion 41 is not limited to this configuration, and at least one of the sealing film 210, the second insulating layer 270, and the second substrate 50 may be removed compared with the first portion 40. The first wiring lines 26 on the first substrate 21 are coupled to the detection circuit 48 included in the control circuit 122 via the signal lines SL at the other end 72 of the flexible printed circuit board 70 (refer to FIG. 3). The detection circuit 48 is electrically coupled to the lower electrodes 11 of the first and the second optical sensors 10A and 10B via the signal lines SL.

The second substrate 50 has a fourteenth side surface 50m, a fifteenth side surface 50p, a sixteenth side surface 50r, a seventeenth side surface 50q, and an eighteenth side surface 50s in the coupling area AB′.

The first substrate 21 has no side surface at portions overlapping the fourteenth side surface 50m, the fifteenth side surface 50p, the sixteenth side surface 50r, the seventeenth side surface 50q, and the eighteenth side surface 50s.

The second side surface 50b and the second side surface 21b are planar surfaces parallel to each other, but the second side surface 50b is smaller in length than the second side surface 21b. The twelfth side surface 50n and the seventh side surface 21n are planar surfaces parallel to each other, but the length on the coupling area AB′ side of the twelfth side surface 50n is smaller than the length on the coupling area AB′ side of the seventh side surface 21n.

Therefore, the area of the second substrate 50 on the first substrate 21 is smaller than that of the first substrate 21 even on the coupling area AB′ side.

In the peripheral area AC, the first wiring lines 26 and the second wiring line 260 intersect the second side 422. The first wiring lines 26 do not intersect the first side 421 facing the second side 422.

A peripheral area AC′ between the coupling area AB′ and the active area AA has a first portion 44 and a second portion 45. The first portion 44 includes the first insulating layer 27, the sealing film 210, the second insulating layer 270, and the second substrate 50 on the first substrate 21. The second portion 45 is a portion where the sealing film 210, the second insulating layer 270, and the second substrate 50 are removed. The configuration of the second portion 45 is not limited to this configuration, and at least one of the sealing film 210, the second insulating layer 270, and the second substrate 50 may be removed compared with the first portion 44.

Also, in the peripheral area AC′, in the same way as in the peripheral area AC, the first wiring lines 26 intersects a third side 423 that is the boundary between the first portion 44 and the second portion 45, in the second direction Dy. The first wiring lines 26 and the second wiring line 260 intersect the first and the second sides 421 and 422. However, when the detection device 1 is bent toward the third direction, the first wiring lines 26 is less subject to stress on the third side 423. As a result, cracks in the first wiring lines 26 that intersect the first and the third side 423 are reduced.

The third side 423 is provided on the seventeenth side surface 50q. The first wiring lines 26 does not intersect a fourth side 424 that is the boundary between the first portion 44 and the second portion 45 and faces the third side 423. The fourth side 424 is provided on the fifteenth side surface 50p.

A step corresponding to the thickness of the second substrate 50 is formed in the third direction on each of the fourteenth side surface 50m, the sixteenth side surface 50r, and the eighteenth side surface 50s, but the first wiring lines 26 and the second wiring line 260 do not intersect these side surfaces. Therefore, when the detection device 1 is bent toward the third direction, stress generated at the step is difficult to be applied to the first wiring lines 26 and the second wiring line 260.

As a result, when the detection device 1D is bent toward the third direction, stress applied to the first wiring lines 26 is distributed, making the first wiring lines 26 less subject to the stress and reducing cracks in the first wiring lines 26.

### Fifth Embodiment

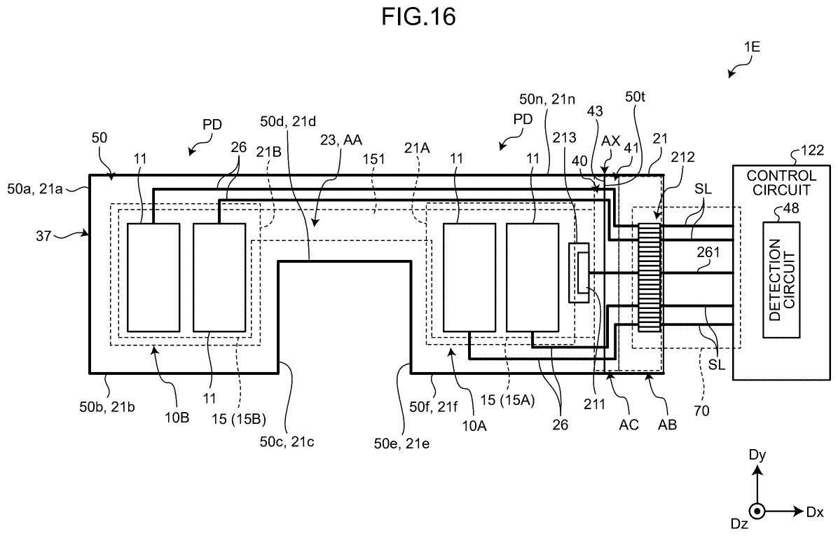

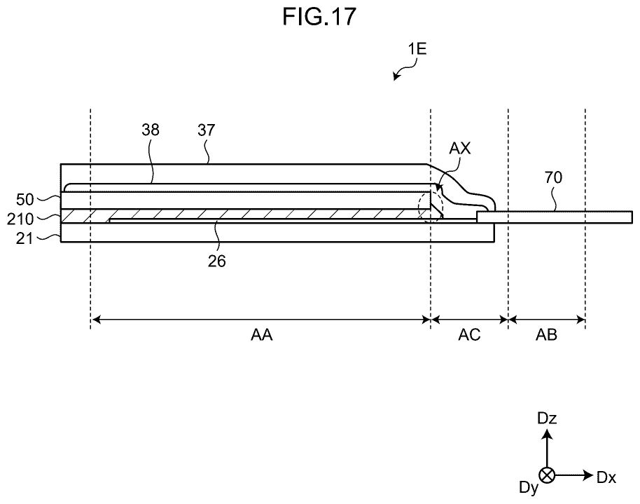

FIG. 16 is a configuration diagram illustrating an exemplary configuration of the first optical sensor and the second optical sensor according to a fifth embodiment. FIG. 17 is a schematic sectional view illustrating an exemplary multilayer configuration of the optical sensor illustrated in FIG. 16. In the following description, the same components as those described in any one of the embodiments described above are denoted by the same reference numerals, and will not be described again. The description of the first portion 40 is the same as that of the first embodiment and is therefore omitted.

As illustrated in FIG. 16, in a detection device 1E according to the fifth embodiment, the first wiring lines 26 intersect the boundary line 43 between the first portion 40 and the second portion 41.

As illustrated in FIG. 17, the area of the second substrate 50 on the upper side is smaller than that of the first substrate 21 on the lower side. As a result, the step AX corresponding to the thickness of the second substrate 50 is formed in the third direction on the flexible printed circuit board 70 side.

The second portion 41 includes the first substrate 21, the first insulating layer 27, the first wiring lines 26, and the second wiring line 260.

As illustrated in FIGS. 16 and 17, a protective film 37 covers the entire active area AA and the peripheral area AC with an adhesive layer 38 interposed therebetween. A portion of the flexible printed circuit board 70 is sandwiched between the protective film 37 and the first substrate 21 with the adhesive layer 38 interposed therebetween. The protective film 37 may extend to the coupling area AB and cover the active area AA, the peripheral area AC, and the coupling area AB with the adhesive layer 38 interposed therebetween.

The protective film 37 is, for example, a film formed into a thin film shape using polyethylene terephthalate (PET) or the like that is a film-like synthetic resin.

The adhesive layer 38 is an adhesive film for adhesively fixing the first substrate 21, the second substrate 50, and the flexible printed circuit board 70 to the protective film 37.

As a result, the rigidity of the step AX is reinforced by the protective film 37. Therefore, when the detection device 1E is bent toward the third direction, the stress is eased and cracks in the first wiring lines 26 can be reduced.

The components in the embodiments described above can be combined as appropriate. Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a photodiode comprising a lower electrode, a lower buffer layer, an active layer, an upper buffer layer, and an upper electrode;
a first substrate;
a first insulating layer located between the first substrate and the photodiode;
a second substrate that covers at least the photodiode so as to sandwich the photodiode between the first substrate and the second substrate, and has an area smaller than the first substrate;
a second insulating layer located between the second substrate and the photodiode;
an active area comprising the active layer of the photodiode;
a coupling area in which a coupling part provided at an end of the first substrate is located;
a peripheral area between the active area and the coupling area;
a sealing film that seals the active area and the peripheral area; and
a first wiring line that couples the lower electrode to the coupling part, wherein
the peripheral area comprises:
a first portion that comprises the first insulating layer, the sealing film, the second insulating layer, and the second substrate on the first substrate; and
a second portion in which at least one of the sealing film, the second insulating layer, and the second substrate is removed compared with the first portion,
a boundary line between the first portion and the second portion extends along a first direction from the coupling area toward the active area, and
the boundary line intersects the first wiring line.

2. The detection device according to claim 1, wherein
the second portion comprises the first insulating layer on the first substrate, with the sealing film, the second insulating layer, and the second substrate removed.

3. The detection device according to claim 1, wherein
the second portion comprises the first insulating layer, the second insulating layer, and the second substrate on the first substrate, with the sealing film removed.

4. The detection device according to claim 1, wherein
the second portion comprises the first insulating layer, the second substrate, and the sealing film on the first substrate, with the second insulating layer removed.

5. The detection device according to claim 1, wherein
the boundary line is parallel to the first direction, and
a direction in which the first wiring line intersecting the boundary line extends is orthogonal to the first direction.

6. The detection device according to claim 1, further comprising another coupling area opposite the coupling area in the first direction.

7. The detection device according to claim 1, wherein the lower electrode and the first wiring line are light-transmitting conductors.

8. The detection device according to claim 1, wherein
the boundary line comprises a first side and a second side that faces the first side, and
the detection device comprises a plurality of the first wiring lines that comprise a wiring line intersecting the first side and a wiring line intersecting the second side.

9. The detection device according to claim 1, comprising a second wiring line that couples the upper electrode to the coupling part, wherein
the boundary line intersects the second wiring line.

10. The detection device according to claim 1, wherein, when the first substrate and the second substrate are viewed along a second direction orthogonal to the first direction, the first substrate and the second substrate are bendable so as to protrude toward a third direction orthogonal to the first direction and the second direction.

11. A detection device comprising:
a photodiode comprising a lower electrode, a lower buffer layer, an active layer, an upper buffer layer, and an upper electrode;
a first substrate;
a first insulating layer located between the first substrate and the photodiode;
a second substrate that covers at least the photodiode so as to sandwich the photodiode between the first substrate and the second substrate, and has an area smaller than the first substrate;
a second insulating layer located between the second substrate and the photodiode;
an active area comprising the active layer of the photodiode;
a coupling area in which a coupling part provided at an end of the first substrate is located;
a peripheral area between the active area and the coupling area;
a sealing film that seals the active area and the peripheral area; and
a first wiring line that couples the lower electrode to the coupling part, wherein
the peripheral area comprises:
a first portion that comprises the first insulating layer, the sealing film, the second insulating layer, and the second substrate on the first substrate; and
a second portion comprising the first insulating layer on the first substrate,
a boundary line between the first portion and the second portion intersects the first wiring lines along a second direction intersecting the first direction from the coupling area toward the active area,
a flexible printed circuit board is coupled to the coupling area,
a protective film covers the second substrate so as to overlap an entire surface of the active area and extends to the peripheral area, and
a portion of the flexible printed circuit board is sandwiched between the protective film and the first substrate.

12. The detection device according to claim 11, wherein the protective film, the first substrate, and the second substrate are polyethylene terephthalate.
